(19) United States
(12) Patent Application Publication
(10) Pub. No.: US 2025/0333465 A1
(43) Pub. Date: Oct. 30, 2025

(54) EFFECTIVE TRUNCATED PACAPS FOR DISEASE MODIFYING TREATMENT OF NEURODEGENERATIVE DISEASES

(71) Applicant: ARIZONA BOARD OF REGENTS ON BEHALF OF THE UNIVERSITY OF ARIZONA, Tucson, AZ (US)

(72) Inventors: Robin L. Polt, Tucson, AZ (US); Michael L. Heien, Tucson, AZ (US); John M. Streicher, Tucson, AZ (US); Torsten Falk, Tucson, AZ (US); Minying Cai, Tucson, AZ (US)

(21) Appl. No.: 19/226,039

(22) Filed: Jun. 2, 2025

Related U.S. Application Data
(63) Continuation-in-part of application No. 16/637,702, filed on Feb. 7, 2020, now abandoned.
(63) Continuation-in-part of application No. 18/572,695, filed on Dec. 20, 2023.
(60) Provisional application No. 62/543,152, filed on Aug. 9, 2017.
(60) Provisional application No. 63/214,740, filed on Jun. 24, 2021.

(51) Int. Cl. C07K 14/575; A61K 38/00

(52) U.S. Cl. CPC C07K 14/57563; A61K 38/00

(57) ABSTRACT

Glycopeptide analogs of secretin family peptides, including PACAP and VIP, are described herein. These glycopeptides analogs can have neuroprotective properties and enhanced ability to cross the blood brain barrier (BBB) and/or enhanced stability. These glycosylated peptides can be used as drugs for treatment of CNS disorders, such as Parkinson's disease.

### CROSS-REFERENCE

This application is a continuation-in-part and claims benefit of U.S. patent application Ser. No. 16/637,702 filed Feb. 7, 2020, which is a 371 and claims benefit of International Application No. PCT/US2018/046136 filed Aug. 9, 2018, which claims benefit of U.S. Provisional Application No. 62/543,152, filed Aug. 9, 2017, the specifications of which are incorporated herein in their entirety by reference.

This application is also a continuation-in-part and claims benefit of U.S. patent application Ser. No. 18/572,695 filed Dec. 20, 2023, which is a 371 and claims benefit of International Application No. PCT/US2022/034944 filed Jun. 24, 2022, which claims benefit of U.S. Provisional Application No. 63/214,740, filed Jun. 24, 2021, the specifications of which are incorporated herein in their entirety by reference.

### STATEMENT REGARDING FEDERALLY SPONSORED RESEARCH OR DEVELOPMENT

This invention was made with government support under Grant Nos. NS091238, NS052727 awarded by National Institutes of Health, and 0607917 awarded by NSF. The government has certain rights in the invention.

### REFERENCE TO AN ELECTRONIC SEQUENCE LISTING

The contents of the electronic sequence listing (name of the file: UNIA 17_35 PCT-US CIP.xml; Size: 95,212 bytes; and Date of Creation: Monday, Jun. 2, 2025) is herein incorporated by reference in its entirety.

### FIELD OF THE INVENTION

The present invention relates to glycosylated analogs of secretin family peptides, including PACAP and VIP, for use as neuroprotective agents.

### BACKGROUND OF THE INVENTION

G-protein coupled receptors (GPCRs) are the largest class of transmembrane proteins, which are involved in mediating a myriad of biological processes, making them popular drug targets. In fact, around 40% of drugs currently on the market target a GPCR. Pituitary adenylate cyclase-activating polypeptide type I receptor (PAC₁) and vasoactive intestinal peptide receptors, VPAC₁ and VPAC₂, are receptors of the secretin family of GPCRs. These three receptors are pleiotropic and widely distributed in the central nervous system (CNS) and periphery.

Pituitary adenylate cyclase activating peptide (PACAP1), which has the sequence: HSDGIFTDSY₁₀SRYRKQMAVK₂₀KYLAAVL (SEQ ID NO: 1), can bind to and activate the PAC₁, VPAC₁, and VPAC₂ receptors. Vasoactive intestinal peptide (VIP) is a 28 amino acid peptide with the sequence: HSDAVFTDNY₁₀TRLRKQMAVK₂₀KYLNSILN (SEQ ID NO: 2), which has 68% identity with PACAP₁₋₂₇, and activates the VPAC₁ and VPAC₂ receptors equally to the PACAP ligand, but is several orders of magnitude less active at the PAC, receptor. These peptide neuromodulators are potentially neurotrophic and/or neuroprotective peptides for treatment of CNS conditions, such as, for example, Parkinson's disease (PD), which is an idiopathic neurological disorder in which the dopaminergic neurons of the substantia nigra pars compacta (SNc) degenerate.

The blood-brain barrier (BBB) prevents entry into the brain of many drugs from the blood. Thus, the presence of the BBB can make the development of new treatments for brain diseases difficult. Glycosylation of peptide chains can improve penetration of the BBB to facilitate cerebral entry of the glycopeptide derivative and, consequently, activation of the target receptor. It is possible that both PACAP and VIP interact strongly with biological membranes, and likely promote the kinetics of binding once the glycosylated peptides arrive at the neuronal membrane by reducing the 3-dimensional search for the receptors to a 2-dimensional “membrane search”. This glycosylation approach may be promising for delivering PACAP and VIP drugs to the central nervous system (CNS) for the treatment of Parkinson's disease (PD).

The present invention features glycosylated peptide analogs of PACAP₁₋₂₇ or VIP₁₋₂₈ that have enhanced ability to cross the BBB and/or enhanced half-lives, and that can target specific GPCRs both in and outside the CNS to treat several conditions, such as, for example, Parkinson's disease.

### SUMMARY OF THE INVENTION

It is an objective of the present invention to provide for glycopeptides that target the PAC1, VPAC₁, and VPAC₂ receptors, along with methods of use thereof in treating CNS disorders, such as Parkinson's disease, as specified in the independent claims. One of the unique and inventive technical features of the present invention is the use of carbohydrates to modulate the amphipathicity of various peptides, ultimately enhancing their BBB penetration, bioavailability, and enzymatic stability. Embodiments of the invention are given in the dependent claims. Embodiments of the present invention can be freely combined with each other if they are not mutually exclusive.

According to some aspects, the present invention features a glycopeptide analog of

PACAP₁₋₂₇
(SEQ ID NO: 1)

VIP₁₋₂₈
(SEQ ID NO: 2)

In some embodiments, the glycopeptide analog may comprise a sequence according to any one of the following:

(SEQ ID NO: 3)
HSDGIFTDSY₁₀SRYRKQX¹AVK₂₀KYLAAVX²;

or

(SEQ ID NO: 4)
HSDAVFTDNY₁₀TRLRKQX¹AVK₂₀KYLNSILN.

In some embodiments, X₁ may be M or norleucine or norvaline. Without wishing to be bound by a theory, when X¹ is nordeucine, the glycopeptide analog can have an increased stability as compared to the glycopeptide analog where X¹ is M. In other embodiments, X² may be L or S. In preferred embodiments, at least one of the S residues in the sequence may be glycosylated with a glycan. In other embodiments, the glycopeptide analog may comprise a sequence according to any one of the following: SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, and SEQ ID NO: 52.

According to further embodiments, the present invention may also feature a composition comprising a glycopeptide, said glycopeptide comprises a peptide sequence consisting of 5 to 20 amino acid residues and having at least 80% sequence identity to SEQ ID NO: 35. In some embodiments, at least one amino acid residue within the sequence is glycosylated. In some embodiments, the peptide comprises a norleucine residue. In other embodiments, the peptide comprises a norvaline residue.

In certain embodiments, the present invention features a composition comprising a glycopeptide, said glycopeptide comprises a peptide sequence consisting of 20 amino acid residues and having at least 80% sequence identity to SEQ ID NO: 35. In other embodiments, the composition comprises a glycopeptide, said glycopeptide comprises a peptide sequence consisting of 15 amino acid residues and having at least 80% sequence identity to SEQ ID NO: 35. In some embodiments, the composition comprises a glycopeptide, said glycopeptide comprises a peptide sequence consisting of 11 amino acid residues and having at least 80% sequence identity to SEQ ID NO: 35. In further embodiments, the composition comprises a glycopeptide, said glycopeptide comprises a peptide sequence consisting of 9 amino acid residues and having at least 80% sequence identity to SEQ ID NO: 35. In some embodiments, at least one amino acid residue within the sequence is glycosylated. In some embodiments, the peptide comprises a norleucine residue. In other embodiments, the peptide comprises a norvaline residue. In some embodiments, the peptide has a sequence according to SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, or SEQ ID NO: 52.

In some embodiments, the glycan is a saccharide. In some embodiments, the said saccharide is a monosaccharide, a disaccharide, a trisaccharide, a polysaccharide, or a combination thereof. The saccharide may be selected from the group consisting of glucose, maltose, lactose, melibiose, maltotriose, sucrose, trehalose, altose, saccharose, cellobiose, gentibiose, isomaltose, primeveose, galactose, xylose, mannose, manosaminic acid, fucose, GalNAc, GlcNAc, idose, iduronic acid, glucuronic acid, sialic acid, and polysaccharides related to the Thompsen-Friedrich antigens (Tn), as well as gangliosides or globosides. In some embodiments, the glycan is an O-linked glycan.

According to another aspect, the present invention may feature glycopeptide analog selected from a group consisting of: SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, and SEQ ID NO: 52.

In various embodiments, the glycosylated peptides described herein are amphipathic and exhibit enhanced biological properties, including an increased ability to cross the blood-brain barrier (BBB) relative to their unglycosylated counterparts.

Additionally, the glycopeptide analogs may function as PAC₁ or VPAC₁ receptor agonists, or as VPAC₂ receptor antagonists, thereby enabling diverse receptor-specific activities depending on their structural and chemical modifications.

According to other aspects, the glycopeptide analogs described herein may be utilized in pharmaceutical formulations and methods of treatment. In some embodiments, pharmaceutical compositions comprising the glycopeptide analogs may be effective for treating symptoms associated with Parkinson's disease. In other lycopepti, pharmaceutical compositions comprising the glycopeptides analogs may be effective for treating or preventing symptoms associated with degeneration of dopaminergic neurons of the substantia nigra pars compacta.

According to some other aspects, the present invention may feature a method of treating a symptom associated with Parkinson's disease in a subject. In other aspects, the present invention may feature a method of treating a symptom associated with degeneration of dopaminergic neurons of the substantia nigra pars compacta. Said methods may comprise administering to the subject a therapeutically effective amount of a composition comprising any of the glycopeptide analogs described herein, thereby alleviating the symptom.

Any feature or combination of features described herein are included within the scope of the present invention, provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE DRAWINGS

The features and advantages of the present invention will become apparent from a consideration of the following detailed description presented in connection with the accompanying drawings in which:

FIGS. 1A, 1B, and 1C show binding assay for the truncations of PACAP peptides with PACAP receptors, i.e., PAC₁ (FIG. 1A), VPAC₁ (FIG. 1B), and VPAC₂ (FIG. 1C) via Plasmon Wave Guide Resonance Spectroscopy (PWR). Angle Shift indicates the peptide induced conformational changes.

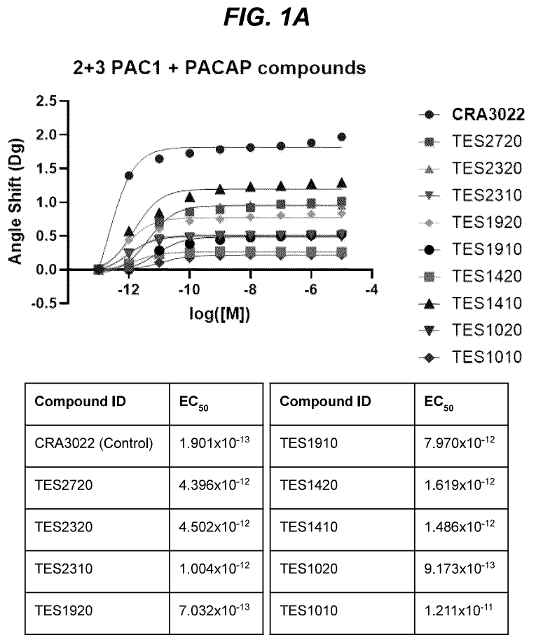

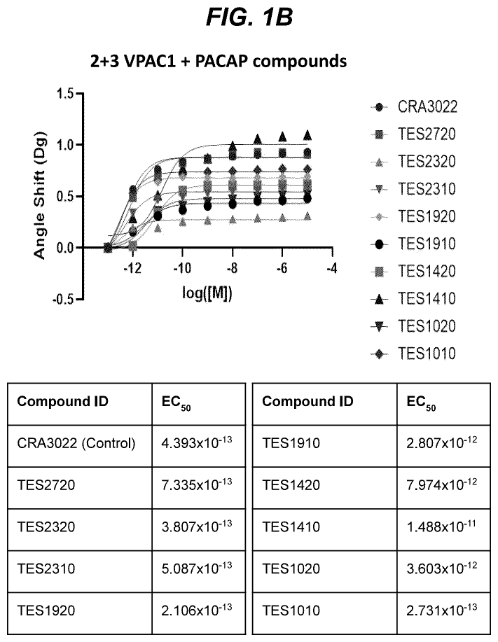

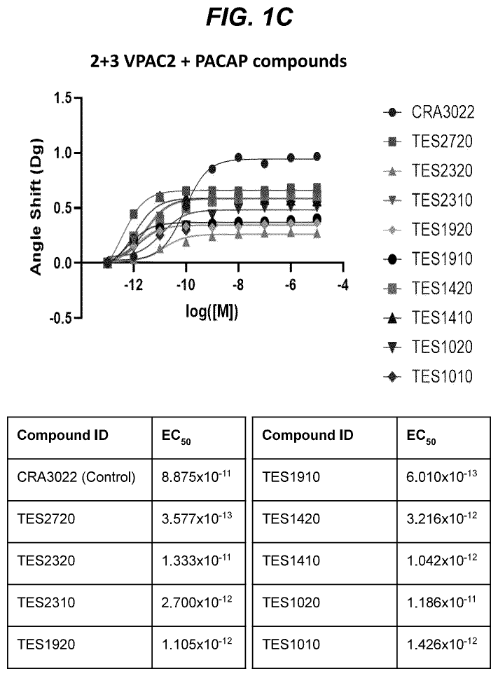

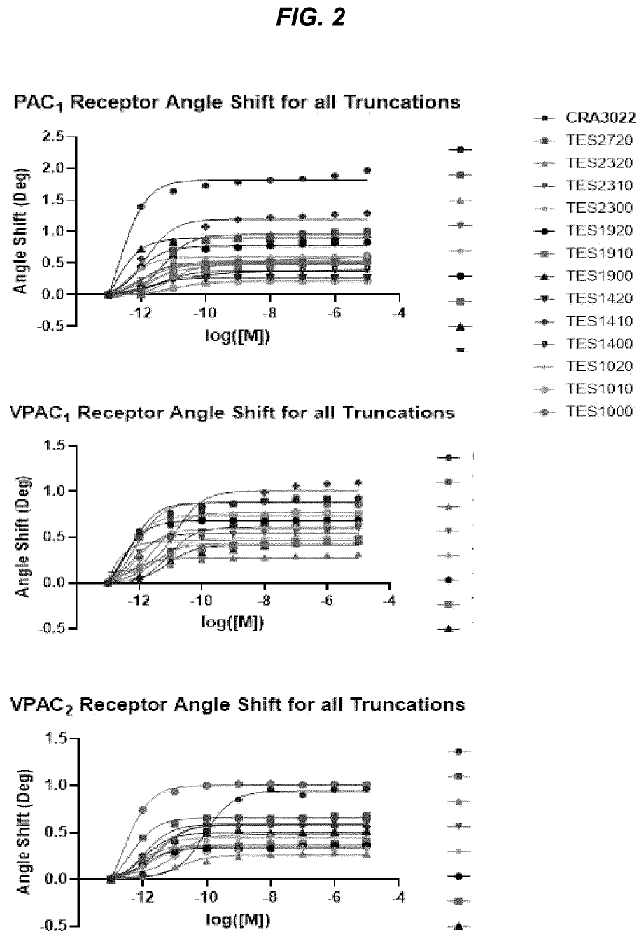

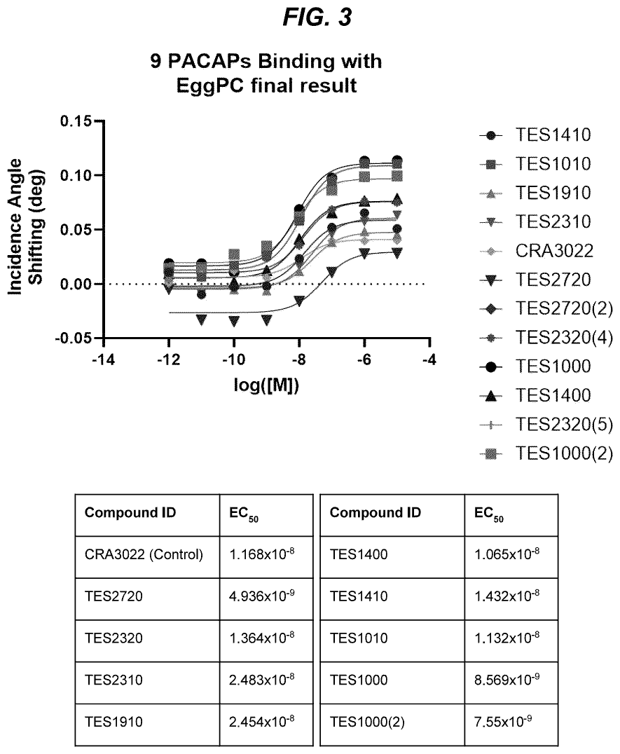

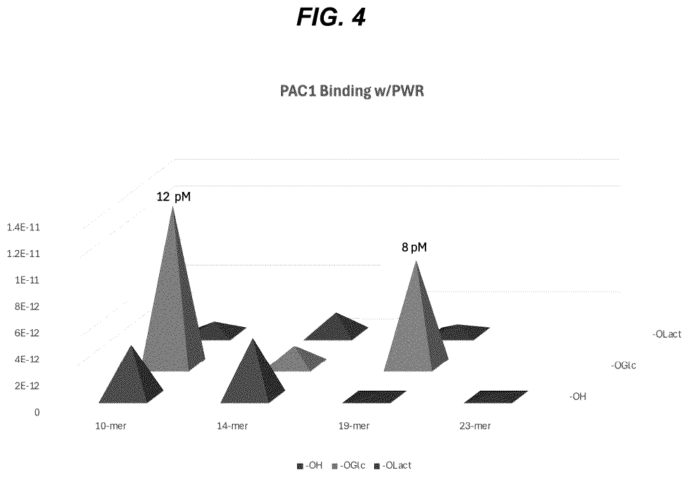

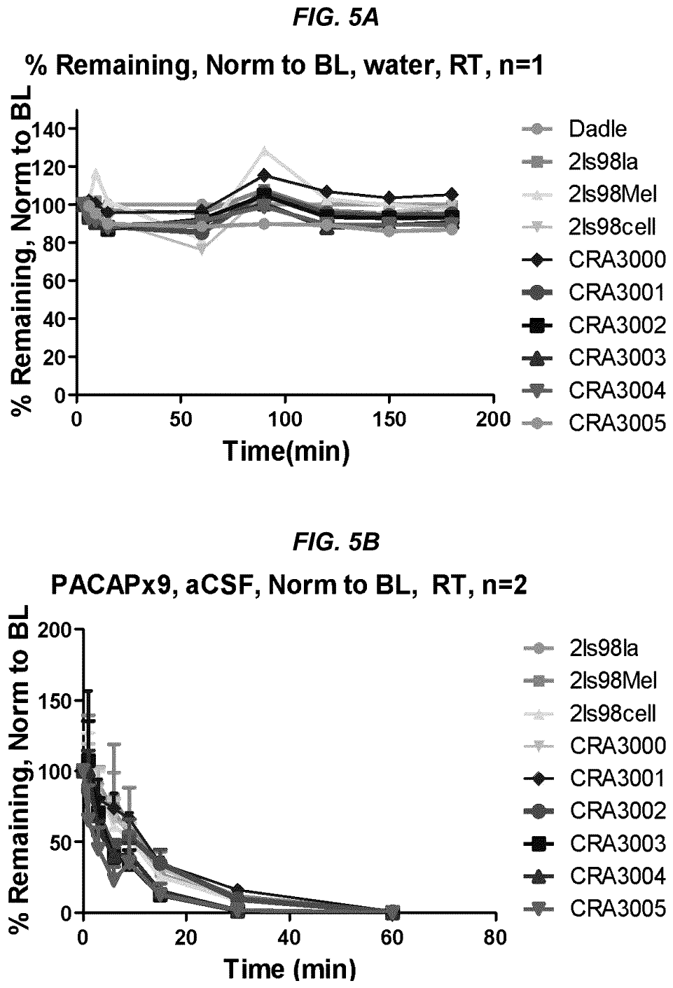

TABLE 1
Sequences in FIGs. 1A-1C.

| Compound | SEQ ID | |
|---|---|---|
| ID | NO: | Sequence |
| | | 10 20 |
| PACAP | 1 | HSDGIFTDSY SRYRKQMAVK KYLAAVL |
| TES2700 | 26 | HSDGIFTDSY SRYRKQÑAVK KYLAAVLS |
| TES2710 | 27 | HSDGIFTDSY SRYRKQÑAVK KYLAAVLS* |
| TES2720 | 28 | HSDGIFTDSY SRYRKQÑAVK KYLAAVLS** |
| TES2711 | 29 | HSDGIFTDSY SRYRKQÑAVK KYLS*AVLS* |
| 23-mers | | 10 20 |
| TES2300 | 30 | HSDGIFTDSY SRYRKQÑAVK KYLS |
| TES2310 | 31 | HSDGIFTDSY SRYRKQÑAVK KYLS* |
| TES2320 | 32 | HSDGIFTDSY SRYRKQÑAVK KYLS** |
| TES2312 | 33 | HSDGIFTDSY SRYRKQÑAVK KYLS*S* |
| TES2311 | 34 | HSDGIFTDSY SRYRKQÑAVS* KYLS* |
| 19-mers | | 10 20 |
| TES1900 | 35 | HSDGIFTDSY SRYRKQÑAVS |
| TES1910 | 36 | HSDGIFTDSY SRYRKQÑAVS* |
| TES1920 | 37 | HSDGIFTDSY SRYRKQÑAVS** |
| TES1912 | 38 | HSDGIFTDSY SRYRKQÑAVS*S* |
| TES1911 | 39 | HSDGIFTDSY SRYRS*QÑAVS* |
| 14-mers | | 10 |
| TES1400 | 40 | HSDGIFTDSY SRYRS |
| TES1410 | 41 | HSDGIFTDSY SRYRS* |
| TES1420 | 42 | HSDGIFTDSY SRYRS** |
| TES1412 | 43 | HSDGIFTDSY SRYRS*S* |
| TES1411 | 44 | HSDGIFTDSY S*RYRS* |
| 10-mers | | |
| TES1000 | 45 | HSDGIFTDSYS |
| TES1010 | 46 | HSDGIFTDSYS* |
| TES1020 | 47 | HSDGIFTDSYS** |
| TES1012 | 48 | HSDGIFTDSYS*S* |
| 8-mers | | |
| TES0800 | 49 | HSDGIFTDS |
| TES0810 | 50 | HSDGIFTDS* |
| TES0820 | 51 | HSDGIFTDS** |
| TES0812 | 52 | HSDGIFTDS*S* |
| | | Ñ = Norleucine |
| | | S* = glycosylated S (L-Serine-β-D-Glucoside) |
| | | S** = lactosylated S (L-Serine-β-D-Lactoside) |
| TESXXX | | N-Acylation can be done with the active glycopeptides. |
| | | Based on the binding and transport results from the first 6 |
| | | glycopeptides, modifications can also be made by the |
| | | substitution of p-F-Phe(6), A(7), homo-Phe(6), Hyp(2) in |
| | | addition to the original modifications. |

FIG. 2 shows binding assays for all truncations of PACAP peptides with PACAP receptors via Plasmon Wave Guide Resonance Spectroscopy (PWR). Angle Shift indicates the peptide induced conformational changes.

FIG. 3 shows the results of glycopeptide binding to purified hen egg phosphatidylcholine (PC) membranes, as measured by plasmon waveguide resonance (PWR). Several glycopeptides were tested, and dissociation constants (Kd values) were determined. All compounds demonstrated nanomolar affinity for the PC membranes. Notably, even the highly truncated glycopeptide TES1010 and its unglycosylated analogue TES1000 exhibited strong interactions with the lipid bilayer, indicating that membrane association is retained despite significant sequence truncation.

FIG. 4 shows that truncation of the unglycosylated 23-mer peptide to 19, 14, and 10 amino acids preserves binding affinity to the PAC1 receptor, as measured by PWR analysis. This effect is observed consistently across peptides that are unglycosylated (Ser28-OH), glycosylated with a glucoside (SerXX-O-β-D-Glc), or glycosylated with a lactoside (SerXX-β-Lact). Among the analogues tested, those with the lowest measured affinities still exhibited binding in the low picomolar range (12 pM and 8 pM), indicating that the pharmacophore tolerates both truncation and glycosylation without complete loss of receptor engagement.

FIG. 5A shows in vitro stability data for the PACAP analogs of the present invention in aqueous solution. The peptides are stable over hours. The sequences 2ls98la, 2ls98Mel, 2ls98cell, CRA3000, CRA3001, CRA3002, CRA3003, CRA3004, CRA3005 corresponds to the lactoside, melibiose, and cellobioside modified versions of PACAP. DADLE is a non-endogenous peptide used as a control. The sequences in FIG. 5A are shown in TABLE 2 below.

FIG. 5B shows the stability in cerebral spinal fluid. The compounds break down at various rates corresponding to their modifications.

TABLE 2
Sequences in FIGs. 5A-5B

| Compound | SEQ ID | |
|---|---|---|
| ID | NO: | Sequence |
| | | 10 20 |
| PACAP₂₇ | 1 | HSDGIFTDSY SRYRKQMAVK KYLAAVL |
| 2ls98LAC | 17 | HSDGIFTDSY SRYRKQLAVK KYLAAVL-Ser(Lactose) |
| 2ls98MEL | 18 | HSDGIFTDSY SRYRKQLAVK KYLAAVL-Ser(Melibiose) |
| 2ls98CEL | 19 | HSDGIFTDSY SRYRKQLAVK KYLAAVL-Ser(Cellobiose) |
| CRA3000 | 20 | HSDGIFTDSY SRYRKQÑAVK KYLAAVLŚ |
| CRA3001 | 21 | HsDGIFTDSY SRYRKQÑAVK KYLAAVŚ |
| CRA3002 | 22 | HsDGIFTDSY SRYRKQÑAVK KYLAAŚL |
| CRA3003 | 23 | HsDGIFTDSY SRYRKQÑAVK KYLAAVLS̆ |
| CRA3004 | 24 | HSDGIFTDSY SRYRKQÑAVK KYLAAVS̆ |
| CRA3005 | 25 | HsDGIFTDSY SRYRKQÑAVK KYLAAS̆L |
| | | s = D-Serine |
| | | Ñ = Norvaline |
| | | Ś = L-Serine-β-D-Glucoside |
| | | S̆ = L-Serine-β-D-Lactoside |
| CRAXXX | | N-Acylation can be done with the active glycopeptides. |
| | | Based on the binding and transport results from the first 6 |
| | | glycopeptides, modifications can also be made by the |
| | | substitution of p-F-Phe(6), A(7), homo-Phe(6), Hyp(2) in |
| | | addition to the original modifications. |

### DESCRIPTION OF PREFERRED EMBODIMENTS

As used herein, the natural amino acids refer to the twenty amino acids that are found in nature, i.e., occur naturally. The natural amino acids are as follows: alanine, arginine, glycine, asparagine, aspartic acid, cysteine, glutamine, glutamic acid, serine, threonine, histidine, lysine, methionine, proline, valine, isoleucine, leucine, tyrosine, tryptophan, and phenylalanine. This application adheres to the IUPAC rules of standard abbreviations for amino acids.

As used herein, the term “unnatural amino acids” refers to amino acids that are not naturally encoded or found in the genetic code of any organisms. Typically, the unnatural amino acids are different from the twenty naturally occurring amino acids in their side chain functionality. A non-limiting example of an unnatural amino acid is Norieucine (Nie).

Each amino acid may be either natural or unnatural of the “D” or “L” configuration which corresponds to the stereochemical designation “S” and “R,” respectively. As known to one of ordinary skill in the art, only L-amino acids are manufactured in cells and incorporated into proteins. The letter “D” preceding any abbreviation for an amino acid denotes the D-form of the amino acid, and a lack thereof refers to the L-form, unless specifically stated otherwise.

As defined herein, the term “agonist” refers to a compound that enhances a response. The agonist binds to the same site as the endogenous compound and produces the same type of signal, usually of equal or greater magnitude than the endogenous agent. As defined herein, the term “antagonist” refers to a compound that binds to the same site as the endogenous compound and diminishes or blocks the signal generated by the endogenous agent.

As used herein, the term “glycoside” is defined a molecule formed by a carbohydrate or a saccharide bound to another reactive functional group via a glycosidic bond, which is a covalent bond formed between the hemiacetal group of the carbohydrate and the reactive functional group, such as the hydroxyl group, of another compound.

Glycosylation processes and glycans are well known to one of ordinary skill in the art. In some embodiments, the glycan is branched. In some embodiments, the glycan is unbranched. In some embodiments, the glycan is an N-linked glycan, or an O-linked glycan, or a C-linked glycan, or an S-linked glycan. Examples of glycans include, but are not limited to, glucose, linear or branched trisaccharides of glucose, lactose, maltose, cellobiose, melibiose, melibiose, glucosamine, N-acetylglucosamine, galactose, galactosamine, N-acetylgalactosamine, mannose, mannosamine, N-acetylmannos-amine, xylose, fucose, rhamnose, N-acetylneuraminic acid, N-glycolylneuraminic acid, 2-keto-3-deoxynononic acid, iduronic acid, and glucuronic acid. The present invention is not limited to the aforementioned glycans. For example, the glycan may be selected from all mono-, di-, tri- and poly-saccharides.

In some embodiments, the present invention may also feature a composition comprising a glycopeptide, said glycopeptide comprises a peptide sequence consisting of 5 to 20 amino acid residues and having at least 80% sequence identity to SEQ ID NO: 35. In other embodiments, the glycopeptide comprises a peptide sequence consisting of 5 to 20 amino acid residues and having at least 85% sequence identity to SEQ ID NO: 35. In some embodiments, glycopeptide comprises a peptide sequence consisting of 5 to 20 amino acid residues and having at least 90% sequence identity to SEQ ID NO: 35. In further embodiments, glycopeptide comprises a peptide sequence consisting of 5 to 20 amino acid residues and having at least 95% sequence identity to SEQ ID NO: 35. In some embodiments, the peptide sequences consist of 20, 15, 11, or 9 amino acid residues. In some embodiments, at least one amino acid residue within the sequence is glycosylated. In some embodiments, the peptide comprises a norleucine residue. In other embodiments, the peptide comprises a norvaline residue.

In certain embodiments, the present invention features a composition comprising a glycopeptide, said glycopeptide comprises a peptide sequence consisting of 20 amino acid residues and having at least 80% sequence identity to SEQ ID NO: 35. In other embodiments, the glycopeptide comprises a peptide sequence consisting of 20 amino acid residues and having at least 85% sequence identity to SEQ ID NO: 35. In some embodiments, glycopeptide comprises a peptide sequence consisting of 20 amino acid residues and having at least 90% sequence identity to SEQ ID NO: 35. In further embodiments, glycopeptide comprises a peptide sequence consisting of 20 amino acid residues and having at least 95% sequence identity to SEQ ID NO: 35. In some embodiments, at least one amino acid residue within the sequence is glycosylated. In some embodiments, the peptide comprises a norleucine residue. In other embodiments, the peptide comprises a norvaline residue. In some embodiments, at least one amino acid residue within the sequence is glycosylated. In some embodiments, the peptide comprises a norleucine residue. In other embodiments, the peptide comprises a norvaline residue. In some embodiments, the peptide has a sequence according to SEQ ID NO: 35, SEQ ID NO: 36, SEQ ID NO: 37, SEQ ID NO: 38, or SEQ ID NO: 39.

In further embodiments, the present invention features a composition comprising a glycopeptide, said glycopeptide comprises a peptide sequence consisting of 15 amino acid residues and having at least 80% sequence identity to SEQ ID NO: 35. Alternatively, the glycopeptide may comprise a peptide sequence consisting of 15 amino acid residues and having at least 80% sequence identity to SEQ ID NO: 40. In some embodiments, the glycopeptide comprises a peptide sequence consisting of 15 amino acid residues and having at least 87% sequence identity to SEQ ID NO: 40. In other embodiments, the glycopeptide comprises a peptide sequence consisting of 15 amino acid residues and having at least 93% sequence identity to SEQ ID NO: 40. In some embodiments, at least one amino acid residue within the sequence is glycosylated. In some embodiments, the peptide comprises a norleucine residue. In other embodiments, the peptide comprises a norvaline residue. In some embodiments, the peptide has a sequence according to SEQ ID NO: 40, SEQ ID NO: 41, SEQ ID NO: 42, SEQ ID NO: 43, or SEQ ID NO: 44.

In other embodiments, the present invention features a composition comprising a glycopeptide, said glycopeptide comprises a peptide sequence consisting of 11 amino acid residues and having at least 80% sequence identity to SEQ ID NO: 35. Alternatively, the glycopeptide comprises a peptide sequence consisting of 11 amino acid residues and having at least 80% sequence identity to SEQ ID NO: 45. In some embodiments, the glycopeptide comprises a peptide sequence consisting of 11 amino acid residues and having at least 90% sequence identity to SEQ ID NO: 45. In some embodiments, at least one amino acid residue within the sequence is glycosylated. In some embodiments, the peptide comprises a norleucine residue. In other embodiments, the peptide comprises a norvaline residue. In some embodiments, the peptide has a sequence according to SEQ ID NO: 45, SEQ ID NO: 46, SEQ ID NO: 47, or SEQ ID NO: 48.

In some embodiments, the present invention features a composition comprising a glycopeptide, said glycopeptide comprises a peptide sequence consisting of 9 amino acid residues and having at least 80% sequence identity to SEQ ID NO: 35. Alternatively, the glycopeptide comprises a peptide sequence consisting of 9 amino acid residues and having at least 80% sequence identity to SEQ ID NO: 49. In some embodiments, the glycopeptide comprises a peptide sequence consisting of 9 amino acid residues and having at least 78% sequence identity to SEQ ID NO: 49. In some embodiments, the glycopeptide comprises a peptide sequence consisting of 9 amino acid residues and having at least 89% sequence identity to SEQ ID NO: 49. In some embodiments, at least one amino acid residue within the sequence is glycosylated. In some embodiments, the peptide comprises a norleucine residue. In other embodiments, the peptide comprises a norvaline residue. In some embodiments, the peptide has a sequence according to SEQ ID NO: 49, SEQ ID NO: 50, SEQ ID NO: 51, or SEQ ID NO: 52.

Furthermore, according to one embodiment, the present invention features a glycopeptide analog of PACAP₁₋₂₇ (SEQ ID NO: 1) or VIP₁-2₈ (SEQ ID NO: 2). In some embodiments, the glycopeptide analog may comprise a sequence according to any one of the following:

HSDGIFTDSY₁₀SRYRKQX¹AVK₂₀KYLAAVX² (SEQ ID NO: 3); or

HSDAVFTDNY₁₀TRLRKQX¹AVK₂₀KYLNSILN (SEQ ID NO: 4);

In some embodiments, X¹ may be M or nordeucine, or norvaline. Without wishing to be bound by a theory, when X¹ is nordeucine, the glycopeptide analog can have an increased stability as compared to the glycopeptide analog where X¹ is M. In other embodiments, X² may be L or S. In some embodiments, the S₂ of SEQ ID NO: 3 or SEQ ID NO: 4 may be in a D or L configuration.

In preferred embodiments, at least one of the S residues in the sequence may be glycosylated with a glycan. For example, S₉ of SEQ ID NO: 3 may be glycosylated. As another example, S₁₁ of SEQ ID NO: 3 may be glycosylated. In one embodiment, X² may be S, and this S₂₇ of SEQ ID NO: 3 may be glycosylated. In another embodiment, S₂₅ of SEQ ID NO: 4 may be glycosylated.

Without wishing to be bound by a theory or mechanism, the glycopeptide analogs have an increased ability to cross a blood brain barrier (BBB) as compared to a peptide lacking glycosylation. Further still, the glycopeptide analog may be amphipathic.

In some embodiments, the glycan is a saccharide, such as a monosaccharide, a disaccharide, a trisaccharide, a polysaccharide, or a combination thereof. The saccharide may be selected from the group consisting of glucose, maltose, lactose, melibiose, maltotriose, sucrose, trehalose, altose, saccharose, cellobiose, gentibiose, isomaltose, primeveose, galactose, xylose, mannose, manosaminic acid, fucose, GalNAc, GlcNAc, idose, iduronic acid, glucuronic acid, sialic acid, and polysaccharides related to the Thompsen-Friedrich antigens (Tn), as well as gangliosides or globosides. In other embodiments, the glycan may be a glucose, maltose, melibiose, lactose, or cellobiose. In still other embodiments, the glycan may be an O-linked glycan. For example, the glycan may be O-linked to the serine by bonding to the hydroxyl group in the side chain of serine.

In one embodiment, the glycopeptide analog may be a PAC₁ agonist. In another embodiment, the glycopeptide analog may be a VPAC₁ agonist. In a further embodiment, the glycopeptide analog may be a VPAC₂ antagonist.

TABLE 3, as well as TABLE 1 above, provides non-limiting examples of the sequences of the glycopeptide analogs of the present invention. In some embodiments, the S₂ of any of the sequences may be in a D or L configuration.

TABLE 3

| SEQ ID NO: | SEQUENCE (S* refers to glycosylated S) |
|---|---|
| SEQ ID NO: 5 | HSDGIFTDS*Y₁₀SRYRKQMAVK₂₀KYLAAVL |
| SEQ ID NO: 6 | HSDGIFTDSY₁₀S*RYRKQMAVK₂₀KYLAAVL |
| SEQ ID NO: 7 | HSDGIFTDSY₁₀SRYRKQ-Nle-AVK₂₀KYLAAVL |
| SEQ ID NO: 8 | HSDGIFTDS*Y₁₀SRYRKQ-Nle-AVK₂₀KYLAAVL |
| SEQ ID NO: 9 | HSDGIFTDSY₁₀S*RYRKQ-Nle-AVK₂₀KYLAAVL |
| SEQ ID NO: 10 | HSDGIFTDSY₁₀SRYRKQ-Nle-AVK₂₀KYLAAVS |
| SEQ ID NO: 11 | HSDGIFTDS*Y₁₀SRYRKQ-Nle-AVK₂₀KYLAAVS |
| SEQ ID NO: 12 | HSDGIFTDSY₁₀S*RYRKQ-Nle-AVK₂₀KYLAAVS |
| SEQ ID NO: 13 | HSDGIFTDSY₁₀SRYRKQ-Nle-AVK₂₀KYLAAVS* |
| SEQ ID NO: 14 | HSDAVFTDNY₁₀TRLRKQMAVK₂₀KYLNS*ILN |
| SEQ ID NO: 15 | HSDAVFTDNY₁₀TRLRKQ-Nle-AVK₂₀KYLNSILN |
| SEQ ID NO: 16 | HSDAVFTDNY₁₀TRLRKQ-Nle-AVK₂₀KYLNS*ILN |

It may be appreciated that the glycopeptide analogs of the present invention can be utilized in pharmaceutical formulations and methods of treatment. Thus, in some aspects, the present invention provides for pharmaceutical compositions that comprise any one of the glycopeptide analogs described herein, and methods of use thereof.

In one embodiment, the pharmaceutical composition may be effective for treating or preventing symptoms associated with Parkinson's disease. In another embodiment, the pharmaceutical composition may be effective for treating or preventing symptoms associated with degeneration of dopaminergic neurons of the substantia nigra pars compacta. In preferred embodiments, the pharmaceutical composition may include a therapeutically effective amount of the glycopeptide analog of the invention. The composition may further comprise a pharmaceutically acceptable carrier. In some embodiments, the glycopeptide analog may be present in an amount ranging from 0.001 to 1.0 wt % of the composition. In exemplary embodiments, the composition may be in the form of a tablet, a nasal spray, or an intravenous solution.

According to some aspects, the present invention may feature a method of treating or preventing a symptom associated with Parkinson's disease in a subject. According to other aspects, the present invention may feature a method of treating or preventing a symptom associated with degeneration of dopaminergic neurons of the substantia nigra pars compacta. Said methods may comprise administering to the subject a therapeutically effective amount of a composition comprising any of the glycopeptide analogs described herein. Without wishing to be bound by a theory or mechanism, the glycopeptide analog may be configured to cross through the BBB.

In some embodiments, the composition being administered may further comprise a pharmaceutically acceptable carrier. In other embodiments, the subject may be a mammal, such as a human. In one embodiment, the glycopeptide analog may be administered in a dosage of about 0.001 mg/kg to 100 mg/kg of body weight, or any range in between. In another embodiment, the composition may be administered daily, weekly, or monthly. In further embodiments, the composition is administered intranasally, intravenously, transdermally, or orally.

As used herein, the terms “treat”, “treating”, or “treatment” refer to both therapeutic treatment and prophylactic or preventative measures, with the objective of preventing, reducing, slowing down (lessen), inhibiting, or eliminating an undesired physiological change, symptom, disease, or disorder, such Parkinson's disease. For purposes of this invention, beneficial or desired clinical results include, but are not limited to, alleviation (e.g. reduction, lessening, or inhibition) of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder, as well as those prone to have the condition or disorder, or those in which the condition or disorder is to be prevented or onset delayed. Optionally, the subject or patient may be identified (e.g., diagnosed) as one suffering from the disease or condition (e.g., Parkinson's disease) prior to administration of the peptide analog of the invention. Subjects at risk for Parkinson's disease can be identified by, for example, any or a combination of appropriate diagnostic or prognostic assays known in the art.

A “therapeutically effective amount” refers to an amount that is sufficient to achieve the desired therapeutic result or to have an ameliorating effect on undesired symptoms, but is generally insufficient to cause adverse side effects. The specific therapeutically effective dose level for any particular patient will depend upon a variety of factors including the disease being treated and the severity of the disease; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products.

A “subject” is an individual and includes, but is not limited to, a mammal (e.g., a human, horse, pig, rabbit, dog, sheep, goat, non-human primate, cow, cat, guinea pig, or rodent), a fish, a bird, a reptile or an amphibian. The term does not denote a particular age or sex. Thus, adult and newborn subjects, as well as fetuses, whether male or female, are intended to be included. A “patient” is a subject afflicted with a disease or disorder. The term “patient” includes human and veterinary subjects.

The terms “administering” and “administration” refer to methods of providing a pharmaceutical composition to a subject. Such methods are well known to those skilled in the art and include, but are not limited to, administering the compositions orally, parenterally (e.g., intravenously and subcutaneously), by intramuscular injection, by intraperitoneal injection, intrathecally, transdermally, extracorporeally, topically or the like. Administration of the composition can occur, daily, weekly, monthly, or any period in between. In some embodiments, the composition may be administered periodically for a set period of time, e.g., once per week for between about 1 to 10 weeks. The compound may also be administered chronically throughout a subject's lifetime. One skilled in the art would recognize how to monitor the effectiveness of the treatment and how to adjust the treatment accordingly. A skilled artisan will appreciate that certain factors may influence the dosage and timing required to effectively treat a subject, including but not limited to the severity of the disease or disorder, previous treatments, the general health and/or age of the subject, and other diseases present. If a subject does not respond to the initial dosage and administration of the composition, a person of skill can administer the medication daily for several days until a desired response occurs. A person of skill can monitor a subject's clinical response to the administration of the composition, and administer additional dosages or increase the dosages as needed.

As described above, the compositions can be administered to a subject in a pharmaceutically acceptable carrier. By “pharmaceutically acceptable” is meant a material that is not biologically or otherwise undesirable, i.e., the material may be administered to a subject without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Depending on the intended mode of administration, the pharmaceutical compositions may be in the form of solid, semi-solid or liquid dosage forms, such as, for example, tablets, suppositories, pills, capsules, powders, liquids, suspensions, lotions, creams, gels, or the like, preferably in unit dosage form suitable for single administration of a precise dosage. The compositions will include, as noted above, an effective amount of the selected glycopeptide analog drug in combination with a pharmaceutically acceptable carrier and, in addition, may include other medicinal agents, pharmaceutical agents, carriers, adjuvants, diluents, etc.

In some embodiments, solid compositions may comprise conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, etc., an active compound as described herein and optional pharmaceutical adjuvants in an excipient, such as, for example, water, saline aqueous dextrose, glycerol, ethanol, and the like, to thereby form a solution or suspension. If desired, the pharmaceutical composition to be administered may also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, etc. Actual methods of preparing such dosage forms are known, or will be apparent, to those skilled in this art.

In some embodiments, for oral administration, fine powders or granules may contain diluting, dispersing, and/or surface active agents, and may be presented in water or in a syrup, in capsules or sachets in the dry state, or in a non-aqueous solution or suspension where suspending agents may be included, in tablets where binders and lubricants may be included, or in a suspension in water or a syrup. Where desirable or necessary, flavoring, preserving, suspending, thickening, or emulsifying agents may be included. Tablets and granules may be coated.

Parenteral administration is generally characterized by injection. Common parenteral routes are intramuscular (IM), subcutaneous (SC) and intravenous (IV). Injectables can be prepared in conventional forms, either as liquid solutions or suspensions, solid forms suitable for solution or suspension in liquid prior to injection, or as emulsions. In some embodiments, parental administration may involve use of a slow release or sustained release system, such that a constant level of dosage is maintained.

In other embodiments, the compositions may be administered topically. For topical administration, liquids, suspension, ointments, lotions, creams, gels, drops, suppositories, sprays, powders or the like may be used as long as the active compound can be delivered through the surface of the skin. Conventional pharmaceutical carriers, aqueous, powder or oily bases, thickeners and the like may be necessary or desirable.

### EXAMPLES

The following are non-limiting examples of practicing the present invention. It is to be understood that these examples are not intended to limit the invention in any way, and that equivalents or substitutes are within the scope of the invention.

### Example 1.1: Synthesis of Truncated Peptides and Glycopeptides

Peptide synthesis was performed on a Prelude automated peptide synthesizer. Synthesis was performed either in an automated fashion or semi-manually, where reagents were loaded into the reaction vessels using a syringe. The resin was agitated (i.e., mixed) using a steady flow of argon. The washing steps with dimethylformamide (DMF) and dichloromethane (DCM) were performed for 2 minutes each.

Rink amide resin preparation: 0.5 mmol of Rink Amide-MBHA resin (1 g, ds: 0.5 mmol/g) was placed in a 45 mL reaction vessel and swelled in DMF for 1 hour. Fmoc removal was achieved by the addition of a solution containing 2% DBU-3% piperidine in DMF (10 mL) and mixing for 4 minutes. The mixture was then drained, and the resin was washed once with 10 mL of DMF. Fmoc removal was then repeated for an additional 8 minutes, followed by 6 DMF washes (10 mL, 2 minutes).

Glycosyl amino acid loading: 0.65 mmol, (1.3 molar equivalents) [N-(9-fluorenylmetoxycarbonyl)-L-serne-3-yl]peracetyl-b-O-glycopyranoside and 0.65 mmol, (1.3 molar equivalents) 6-Cl-HOBt were placed into a vial and dissolved in 8 mL N-methyl-2-pyrrolidinone (NMP). 0.65 mmol (1.3 molar equivalents) of diisopropylcarbodiimide (DIC) was then added to the solution. The mixture was vortexed for 1 minute and then added to the resin. The reaction mixture was mixed overnight for 16 hours. The mixture was diluted with DMF (10 mL) and drained immediately. Then, the resin was washed six times with DMF (10 mL) and then four times with DCM. The unreacted NH₂ sites on the resin were then capped with a solution of 10% N,N-diisopropylethylamine and 10% acetic anhydride (Ac₂O) in 8 mL DCM. This reaction was allowed to proceed for 1 hour. The resin was then washed six times with DCM and then washed 4 times with DMF to prepare the resin for the next automated steps.

Prelude automated synthesis: The Leu27-Tyr10 amino acid series was prepared using the automated SPPS feature on the PreludeÓ automated peptide synthesizer. The Fmoc group was removed as described above, and a solution containing the desired Fmoc-amino acid (3 equivalents), HBTU (3 equivalents), and N-methylmorpholine (12 equivalents) was loaded into the resin. The reaction mixture was mixed for 30 minutes, followed by a single DMF wash (10 mL). The coupling reaction was repeated a second time for 30 minutes, and the resin was then washed six times with DMF. Subsequent deprotection and coupling cycles were then performed up to Tyrosine10.

Manual loading of DS dipeptide: The Fmoc group was initially removed as described above. Then, 1 mmol of Fmoc-DS-OH or Fmoc-DG-OH (2 equiv.) and 1 mmol of 6-CI-HOBt (2 equiv.) were added to a vial and dissolved in 8 mL of NMP. 1 mmol of DIC (2 equiv.) was then added to the solution. The mixture was vortexed for 1 minute and then added to the resin. The reaction mixture was mixed for 40 minutes. The resin was then washed once with DMF (10 mL), and a second coupling was performed for 60 minutes. The mixture was diluted with DMF (10 mL) and drained immediately. Then the resin was washed six times with DMF (10 mL)

Automated addition of IFT.: The Ile5-Thr7 amino acid series was prepared using the automated SPPS feature on the PreludeÓ automated peptide synthesizer. The Fmoc group was removed as described above, and a solution containing the desired Fmoc-amino acid (3 equivalents), HATU (3 equivalents), and 2,4,6-trimethylpyridine (12 equivalents) in 10 mL of DMF was loaded to the resin. The reaction mixture was mixed for 30 minutes, followed by a single wash with DMF (10 mL). The coupling reaction was repeated a second time for 30 minutes, and the resin was then washed six times with DMF. Subsequent deprotection and coupling cydes were then performed up to Isoleucine5.

Manual loading of amino acids: The Fmoc group was initially removed as described above. Then, 1.5 mmol amino acid (3 equiv.) and 1.5 mmol of 6-CI-HOBt (3 equiv.) were added to a vial and dissolved in 8 mL of NMP. 1.5 mmol of DIC (3 equiv.) was then added to the solution. The mixture was vortexed for 1 minute and then added to the resin. The reaction mixture was mixed for 40 minutes. The resin was then washed once with DMF (10 mL), and a second coupling was performed for 60 minutes. The mixture was diluted with DMF (10 mL) and drained immediately. Then the resin was washed six times with DMF (10 mL). After the final amino acid, the Fmoc group was initially removed as described above.

Acetyl Cleavage: 120 mL of a 50% solution containing NH₂NH₂×H₂O in NMP (10 mL per reaction vessel) was prepared and added to the resin. The solution was mixed overnight for 16 hours. The solution was then drained, and a second 10 mL portion of 50% NH₂NH₂×H₂O was added to each reaction vessel. This solution was mixed for an additional 2 hours. The 50% NH₂NH₂×H₂O was then drained, and the resin was washed eight times with DMF (10 mL), eight times with DCM (10 mL), and dried under vacuum for 3 hours.

Cleavage from the resin and global side chain deprotection: The dried resin was treated with an acidic cleavage cocktail containing TFA, DCM, H₂O, triethylsilane, and anisole (90:10:2:3:0.5). The resin was mixed for 1 hour, and the solution was collected into a 45 mL centrifuge tube. The cleavage step was repeated two more times for 10-minute periods. The combined fractions slowly evaporated over a stream of argon until the peptide began to crash out. Cold ether (˜40 mL) was then added to precipitate the peptide, and the mixture was centrifuged for 10 minutes at 5 G. The ether layer was decanted off, and ether (˜40 mL) was added to the crude peptide and centrifuged once more. This process was repeated for a third time. After decanting the ether layer, the crude peptide was dried under vacuum overnight.

HPLC purification and characterization of crude peptides: These crude samples were then purified on a Gilson system with a UV detector (at 280 nm) using a Vydak C18 preparative reversed phase column (250 mm×50 mm) using a gradient of 5-80% CH₃CN vs 0.1% CF₃COOH in H₂O over 60 min to give the glycopeptides in pure form, assessed for purity by analytical HPLC (Inspire C18 5 μm 250 mm×4.6 mm column) on a Varian LC with a diode array detector system (at 280 nm) employing the same gradient over a period of 15 min. The pure fractions obtained from preparative HPLC purification were frozen at −80° C. and then lyophilized to afford the pure peptides as white and fluffy solids. The pure peptides were then characterized using mass spectrometry (ESI-MS).

### Example 1.2: Synthesis of Peptides and Glycopeptides

The glycopeptide analogs were synthesized using methods for the incorporation of the glycosides. All of the peptides and glycopeptides used in this study were highly purified (>97% based on HPLC analysis).

Resin preparation. One gram (1.00 g) of Rink amide-MBHA resin (0.5 mmol, substitution=0.5 mmol/g) was placed into a 12 mL fritted syringe. The resin was washed with 6 mL DMF and placed on a slow tumbler for 2 minutes. The DMF was expelled, and the washing was repeated with an additional 6 mL DMF.

Fmoc deprotection. A mixture of organic bases 2% DBU-2% piperidine in DMF (6 mL) was added to the resin and the syringe was tumbled for 5 min. The organic base mixture was expelled and the cleavage treatment was repeated for an additional 10 min with fresh base. The free NH₂ resin was suspended in fresh DMF (6 mL) and the syringe was tumbled for 2 minutes. The DMF was expelled and the washing was repeated 4× with DMF. The resin was washed a final time with NMP the same manner.

Fmoc amino acid glycoside peracetate coupling. 0.65 mmol, (1.3 eq.) [N-(9-fluorenylmetoxycarbonyl)-L-serine-3-yl]peracetyl-β-O-glycopyranoside and 0.65 mmol, (1.3 eq.) HOBT were placed into a 20 mL vial and dissolved in 6 mL NMP. Into the solution 0.65 μmol, (1.3 eq.) DIC was added, and the mixture was shaken for 1 minute, then added to the resin. The syringe with the resin and activated amino acid was tumbled for 5 minutes, then it was placed in the center of the rotating plate of 1250 Watt commercial microwave oven. The oven was set to Power Level 1 (intermittent heating) for 10 minutes. During this time, every 30 seconds, the syringe was shaken manually for 10 seconds and returned to the microwave for a total of 10 minutes. The solvent was expelled from the syringe and the resin was washed once initially with NMP and 5× with DMF in the same manner as described above.

The Fmoc protecting group was removed as described above, and the DMF washing protocol was repeated, ending with the NMP wash.

Fmoc amino acid coupling. Subsequent amino acid couplings were accomplished on the Prelude® synthesizer with 3 eq HBTU, 12 eq of NMM, and 3 eq of the desired amino acid in 14 mL of DMF in double coupling mode. After coupling, the resin was washed 6× with 10 mL DMF for 2 minutes. The Fmoc removal was accomplished as above, but the washing was 6× with 10 mL DMF for 2 minutes. After the last Fmoc cleavage, the acetyl groups were removed.

Acetate cleavage. The wet (DMF) resin was treated with 10 mL of 50% H₂NNH₂·H₂O in DMF for 2×30 minutes, and 1×60 minutes, followed by 6× washes with 10 mL DMF, 2× with 10 mL CH₃OH, 4× with 10 mL DMF and 6× with 10 mL CH₂Cl₂, followed by drying in vacuo.

Peptide cleavage. Each batch of dried peptide resin was cleaved in a fritted syringe in which it was assembled using 10 mL of a “cleavage cocktail” (9.0 mL of TFA, 1.0 mL of CH₂Cl₂, 0.25 mL of Et₃SiH, 0.25 mL of H₂O, and 0.05 mL anisole) for 1 hour at RT. After cleavage the solution was expelled and the resin was washed 2× with 4 mL of the cleavage cocktail, and the combined solutions were concentrated to −5 mL (oil) by a stream of dry N₂. Cold Et₂O (35 mL) was poured over the peptide solutions to precipitate each product. The crude glycopeptides were centrifuged, dried in vacuo, then re-dissolved in H₂O, freeze-dried, analyzed by analytical HPLC and separated by preparative HPLC. The appropriate fractions were freeze-dried to provide amorphous white powders. Yields of the purified peptides/glycopeptides ranged from 14-39%.

Analytical HPLC conditions. Varian ProStar® HPLC system. Mobil phase: 1 mL/min flow rate, gradient 100% solvent A to 100% solvent B over 20 minutes. Solvent A is 5% CH₃CN in H₂O with 0.1% TFA, B solvent is 80% CH₃CN in H₂O with 0.1% TFA. Column: Dikma Technology, Inspire™, C₁₈, 5 μm, 250×4.6 mm. Detection at 280 nm.

Preparative HPLC conditions. Gilson Preparative HPLC system. Mobil phase: 25 mL/min flow rate, gradient 100% A solvent to 100% B solvent over 60 minutes. Solvents A and B are as in the analytical HPLC system. Column: Phenomenex, Luna C₁₈ , 10 μm, 250×50 mm. Detection at 280 nm.

### Example 2: Shotgun Microdialysis

A persistent challenge in animal research is the control for all the variables in complex biological systems; even genetically identical animals do not react identically. Different animals often have different responses to the same treatment, and it is difficult to control for variations in the injection site between animals, often leading to large numbers of animals being used to produce a statistically valid result. We have developed an alternative method that controls for intra-animal and injection site variation and reduces the number of animals required. We have coined this technique “shotgun microdialysis,” in which we inject a single animal with multiple compounds of interest and monitor the CSF concentrations using microdialysis. This allows us to directly compare the compounds within a single animal, with a single injection site, and account for the variability between animals.

In microdialysis, a probe with a semi-permeable membrane is surgically implanted into the target region (the striatum in this case), and the perfusate flows through the probe, allowing molecular species of the appropriate molecular weights (our drug candidates) to diffuse across the membrane as a function of concentration gradient. Microdialysis allows for sampling from a region without altering the volume of that region, which is of particular relevance when studying the CSF

Animals for serum stability in vivo, and CSF microdialysis studies. Male Sprague-Dawley rats (275-325 grams) may be used for in vivo experiments. Animals can be purchased from Harlan Laboratories (Indianapolis, IN) and housed in a temperature and humidity controlled room with 12 h reversed light/dark cycles with food and water available ad libitum. All animals are treated as approved by the Institutional Animal Care and Use Committee, University of Arzona, and in accordance with the NIH Guidelines for the Care and Use of Laboratory Animals. Both the number of animals used and their suffering are minimized.

Compounds are spiked into rat serum at 37° C. and monitored over time. Aliquots are quenched with acetic acid and spiked with internal standard (dAdLE), then desalted with C¹⁸ ZipTips®. Samples are run in duplicate with direct injection using a 20-μL injection loop. Quantification was based on the area under the curve (AUC) for the identified MS2 fragments over the course of the injection.

Probe recovery studies are performed with a series of microdialysis probes to optimize for the best recovery of the compounds. Probes are submerged in a stirring solution of the glycopeptide analog compounds in a CSF, and recovery was calculated by comparing the solution concentration to the concentration of the dialysate, accounting for dilution. As microdialysis is inherently a diffusion-limited technique, there is a delicate balance between probe recovery and temporal resolution. The slower the perfusate is flowed through the probe, the higher the recovery will be, but the sampling time is increased. A flow rate of 0.5 μL/minute may be used to optimize both percent recovery and temporal resolution. The pairing of a second line that introduces a preservation solution can yield a temporal resolution of 10 minutes for the collection of 10 μL of solution. Increasing the flow rate would increase the temporal resolution but would also decrease the percent recovery.

To directly compare the in vivo lifetime and BBB penetration of the glycopeptides analogs, a method that involves dual blood draws and microdialysis may be used. The compounds can be injected intravenously at 10 mg/kg via a single tail vein injection. Blood draws can be taken at t=−10, 1, 5, 10, 20, 30, 40, 50, 60, 70, 80, and 90 minutes (where t=0 corresponds to injection). Microdialysis fractions are time-locked with blood draws at ten-minute increments such that blood draws correlate with the median time of the microdialysis fraction. Blood draws are centrifuged for 2 minutes in a tabletop mini-centrifuge to separate the serum and the red blood cells. Serum is pulled off and diluted 100× into a solution of 50:50 a CSF: “preservation solution” for matrix matching with the dialysate and standards, and must be immediately frozen on dry ice. Dialysate samples are collected for 10 minutes at a flow rate of 0.5 μL/min, with the microdialysis tee coupling a line containing preservation solution to the dialysate line immediately behind the probe, leading to a solution volume of 10 μL after 10 minutes. The dialysate samples must be immediately frozen on dry ice. Post-experiment, samples should be stored in a −80° C. freezer until analysis.

All samples may be desalted using μ-C₁₈ Zip Tips® (EMD Millipore). ZipTip® cleanup is a common practice in proteomics analysis, as it is highly useful for desalting biological samples. However, C₁₈ ZTs may have a poor recovery for our targets of interest, due to their hydrophilicity. In order to improve the recovery but maintain the benefit of desalting, the pairing agent octyl sulfonate (8S) may be added. Octyl sulfonate may increase the recovery of the glycopeptides analogs during the ZipTip® desalting process. Octyl sulfonate can ionically pair with the compounds and its effects can last through ZT and column separations, but is removed during ionization so that the masses of the compounds are not altered.

The serum concentration and CSF concentration estimates can be calculated using a calibration curve, accounting for the dilution factors and the probe recoveries for each experiment. Standards may be matrix-matched to samples and undergo the same sample preparation steps. Probe recoveries for probes used in the in vivo experiments may be calculated by submerging the probe into a vial of standards post-experiment. Note that the probe recoveries are highly variable between probes.

### Example 3: Mass Spectrometric Identification

in vitro studies can be performed using an Applied Biosystems QStar Elite mass spectrometer, using quantitation of MS² fragments. For the in vivo studies, a Proxeon nano-LC coupled to a Thermo LTQ-Orbitrap instrument may be used. MS³ fragments may be quantified for increased specificity. The high number of small peptides present in biological solutions such as blood, paired with the minimal clean-up applied to the method, means that the matrix is highly complex and high specificity is required to differentiate our small peptides from the hundreds that are present. Both retention time and MS³ fragment identification are necessary to assure that the target molecules are being quantified.

### Example 4: Assay Binding of Peptides to PAC and VPAC Receptors

TABLE: 4

| Drug | SEQ ID NO: | Sequence |
|---|---|---|
| PACAP1-27 | 1 | HSDGIFTDSYSRYRKQMAVKKYLAAVL |
| 2ls98Cell | 19 | HSDGIFTDSYSRYRKQLAVKKYLAAV_{d8}L-Ser(Cell) |
| 2ls98Lact | 17 | HSDGIFTDSYSRYRKQLAV_{d8}KKYLAAV_{d8}L-Ser(Lact) |
| 2ls98Mel | 18 | HSDGIFTDSYSRYRKQLAVKKYLAAVL-Ser(Mel) |
| CRA3000 | 20 | HSDGIFTDSYSRYRKQÑAVKKYLAAVL-Ser(Glc) |
| CRA3001 | 21 | HsDGIFTDSYSRYRKQÑAVKKYLAAV-Ser(Glc) |
| CRA3002 | 22 | HsDGIFTDSYSRYRKQÑAVKKYLAAL-Ser(Glc)-L |
| CRA3003 | 23 | HsDGIFADSYSRYRKQÑAVKKYLAAVL-Ser(Glc) |
| CRA3004 | 24 | HsDGIFADSYSRYRKQÑAVKKYLAAV-Ser(Glc) |
| CRA3005 | 25 | HsDGIFADSYSRYRKQÑAVKKYLAAV-Ser(Glc)-L |

TABLE 5

| | PAC1 | | VPAC1 | | VPAC2 | |
|---|---|---|---|---|---|---|
| | EC₅₀ | E_{Max} | EC₅₀ | E_{Max} | EC₅₀ | E_{Max} |
| Drug | (nM) | (%) | (nM) | (%) | (nM) | (%) |
| PACAP1-27 | 0.4, | 100 | 14.8 ± 1.6 | 100 | 0.35 ± 0.16 | 100 |
| | 0.13, | | | | | |
| | 0.34 | | | | | |
| 21s98Cell | 0.84 | 92 | 0.52 | 93 | 55.6 | 91 |
| 21s98Lact | 0.72 | 93 | 0.45 | 101 | 193 | 100 |
| 2ls98Mel | 0.57 | 99 | 0.55 | 102 | 9.4 | 86 |
| CRA3000 | 25.5 | 85 | 1.3 | 90 | 241 | 104 |
| CRA3001 | 54.5 | 86 | 4.8 | 90 | 654 | 107 |
| CRA3002 | >250 | −79 | 5.9 | 93 | >2500 | −95 |
| CRA3003 | >250 | −71 | 78.8 | 93 | >2500 | −42 |
| CRA3004 | NC | NC | 1366 | 85 | NC | NC |
| CRA3005 | NC | NC | 1623 | 78 | NC | NC |

### Example 5: Preparation of a Pharmaceutical Composition

10 g of a glycopeptide analog is mixed with 1900 g of an aqueous solution comprising cellulose, polyvinylpyrrolidone, and sucrose. The glycopeptide analog is according to any one of the sequences described herein. This liquid mixture is passed through granulating sieves and desiccated for at least 24 hours at room temperature to produce a dry mixture. The dry mixture should then be compressed into tablets of desired weight and physical specifications by methods known to those skilled in the art. For instance, the dry mixture is formed into tablets, each weighing 100 mg with an available dose of 0.05 mg of the glycopeptide analog.

### Example 6: Treatment Study of Parkinson's Disease with the Pharmaceutical Composition Described in Previous Examples as Follows

A male Parkinsonian monkey exhibits symptoms of pronounced tremors and bradykinesia. The following treatment is administered to the Parkinsonian monkey: In the first period of treatment, two tablets per day for two weeks are orally administered, followed by a one-week off period, which completes one cycle. After the off period, the cycle is repeated for a total treatment time of 3 months. The Parkinsonian monkey exhibits improvement of motor symptoms and balance and the dyskinesias is significantly reduced. Three independent repetitions of the study are performed.

As used herein, the term “about” refers to plus or minus 10% of the referenced number.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

What is claimed is:

1. A composition comprising a glycopeptide, said glycopeptide comprises a peptide sequence consisting of 5 to 20 amino acid residues and having at least 80% sequence identity to SEQ ID NO: 35, wherein at least one amino acid residue within the sequence is glycosylated.

2. The composition of claim 1, wherein the peptide consists of 20 amino acid residues; wherein the peptide has a sequence according to SEQ ID NO: 35, SEQ ID NO: 36, SEQ ID NO: 37, SEQ ID NO: 38, or SEQ ID NO: 39.

3. The composition of claim 1, wherein the peptide consists of 15 amino acid residues; wherein the peptide has a sequence according to SEQ ID NO: 40, SEQ ID NO: 41, SEQ ID NO: 42, SEQ ID NO: 43, or SEQ ID NO: 44.

4. The composition of claim 1, wherein the peptide consists of 11 amino acid residues; wherein the peptide has a sequence according to SEQ ID NO: 45, SEQ ID NO: 46, SEQ ID NO: 47, or SEQ ID NO: 48.

5. The composition of claim 1, wherein the peptide consists of 9 amino acid residues; wherein the peptide has a sequence according to SEQ ID NO: 49, SEQ ID NO: 50, SEQ ID NO: 51, or SEQ ID NO: 52.

6. The composition of claim 1, wherein the glycan is a saccharide.

7. The composition of claim 1, wherein said saccharide is selected from the group consisting of glucose, maltose, lactose, melibiose, maltotriose, sucrose, trehalose, altose, saccharose, maltose, cellobiose, gentibiose, isomaltose, primeveose, galactose, xylose, mannose, manosaminic acid, fucose, GalNAc, GlcNAc, idose, iduronic acid, glucuronic acid, sialic acid, and polysaccharides related to the Thompsen-Friedrich antigens (Tn), as well as gangliosides or globosides.

8. The composition of claim 6, wherein said saccharide is a monosaccharide, a disaccharide, a trisaccharide, a polysaccharide, or a combination thereof.

9. The composition of claim 1, wherein the glycan is a glucose, a maltose, a melibiose, a lactose, or a cellobiose.

10. The composition of claim 1, wherein the glycan is an O-linked glycan.

11. The composition of claim 1, wherein the glycopeptide is amphipathic.

12. The composition of claim 1, wherein the glycosylated peptide has an increased ability to cross a blood brain barrier (BBB) as compared to a peptide lacking glycosylation.

13. The composition of claim 1, wherein the glycopeptide analog is a PAC₁ agonist.

14. The composition of claim 1, wherein the glycopeptide analog is a VPAC₁ agonist.

15. The composition of claim 1, wherein the glycopeptide analog is a VPAC₂ antagonist.

16. A glycopeptide analog selected from a group consisting of: SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, and SEQ ID NO: 52.

17. The glycopeptide analog of claim 16, wherein the glycan is a saccharide.

18. The glycopeptide analog of claim 16, wherein said saccharide is a monosaccharide, a disaccharide, a trisaccharide, a polysaccharide, or a combination thereof.

19. The glycopeptide analog of claim 16, wherein the glycan is a glucose, a maltose, a melibiose, a lactose, or a cellobiose.

20. The glycopeptide analog of claim 16, wherein the glycan is an O-linked glycan.


SEQUENCE LISTING (52 sequences)

SEQ ID NO: 1 - protein, 27 aa, Homo sapiens
HSDGIFTDSYSRYRKQMAVKKYLAAVL

SEQ ID NO: 2 - protein, 28 aa, Homo sapiens
HSDAVFTDNYTRLRKQMAVKKYLNSILN

SEQ ID NO: 3 - protein, 27 aa, synthetic construct
  REGION 1..27: note = PACAP glycopeptide analog
  SITE 2: note = MISC_FEATURE - D-Ser or L-Ser
  SITE 9: note = MISC_FEATURE - Ser may be glycosylated
  SITE 11: note = MISC_FEATURE - Ser may be glycosylated
  SITE 17: note = MISC_FEATURE - M or Nle
  SITE 27: note = MISC_FEATURE - L or S
  SITE 27: note = MISC_FEATURE - Xaa may be glycosylated Ser
HSDGIFTDSYSRYRKQXAVKKYLAAVX

SEQ ID NO: 4 - protein, 28 aa, synthetic construct
  REGION 1..28: note = VIP glycopeptide analog
  SITE 2: note = MISC_FEATURE - D-Ser or L-Ser
  SITE 17: note = MISC_FEATURE - M or Nle
  SITE 25: note = MISC_FEATURE - Ser may be glycosylated
HSDAVFTDNYTRLRKQXAVKKYLNSILN

SEQ ID NO: 5 - protein, 27 aa, synthetic construct
  REGION 1..27: note = PACAP glycopeptide analog
  SITE 2: note = MISC_FEATURE - D-Ser or L-Ser
  SITE 9: note = MISC_FEATURE - Glycosylated Ser
HSDGIFTDSYSRYRKQMAVKKYLAAVL

SEQ ID NO: 6 - protein, 27 aa, synthetic construct
  REGION 1..27: note = PACAP glycopeptide analog
  SITE 2: note = MISC_FEATURE - D-Ser or L-Ser
  SITE 11: note = MISC_FEATURE - Glycosylated Ser
HSDGIFTDSYSRYRKQMAVKKYLAAVL

SEQ ID NO: 7 - protein, 27 aa, synthetic construct
  REGION 1..27: note = PACAP glycopeptide analog
  SITE 2: note = MISC_FEATURE - D-Ser or L-Ser
  SITE 17: note = MISC_FEATURE - Nle
HSDGIFTDSYSRYRKQXAVKKYLAAVL

SEQ ID NO: 8 - protein, 27 aa, synthetic construct
  REGION 1..27: note = PACAP glycopeptide analog
  SITE 2: note = MISC_FEATURE - D-Ser or L-Ser
  SITE 9: note = MISC_FEATURE - Glycosylated Ser
  SITE 17: note = MISC_FEATURE - Nle
HSDGIFTDSYSRYRKQXAVKKYLAAVL

SEQ ID NO: 9 - protein, 27 aa, synthetic construct
  REGION 1..27: note = PACAP glycopeptide analog
  SITE 2: note = MISC_FEATURE - D-Ser or L-Ser
  SITE 11: note = MISC_FEATURE - Glycosylated Ser
  SITE 17: note = MISC_FEATURE - Nle
HSDGIFTDSYSRYRKQXAVKKYLAAVL

SEQ ID NO: 10 - protein, 27 aa, synthetic construct
  REGION 1..27: note = PACAP glycopeptide analog
  SITE 2: note = MISC_FEATURE - D-Ser or L-Ser
  SITE 17: note = MISC_FEATURE - Nle
HSDGIFTDSYSRYRKQXAVKKYLAAVS

SEQ ID NO: 11 - protein, 27 aa, synthetic construct
  REGION 1..27: note = PACAP glycopeptide analog
  SITE 2: note = MISC_FEATURE - D-Ser or L-Ser
  SITE 9: note = MISC_FEATURE - Glycosylated Ser
  SITE 17: note = MISC_FEATURE - Nle
HSDGIFTDSYSRYRKQXAVKKYLAAVS

SEQ ID NO: 12 - protein, 27 aa, synthetic construct
  REGION 1..27: note = PACAP glycopeptide analog
  SITE 2: note = MISC_FEATURE - D-Ser or L-Ser
  SITE 11: note = MISC_FEATURE - Glycosylated Ser
  SITE 17: note = MISC_FEATURE - Nle
HSDGIFTDSYSRYRKQXAVKKYLAAVS

SEQ ID NO: 13 - protein, 27 aa, synthetic construct
  REGION 1..27: note = PACAP glycopeptide analog
  SITE 2: note = MISC_FEATURE - D-Ser or L-Ser
  SITE 17: note = MISC_FEATURE - Nle
  SITE 27: note = MISC_FEATURE - Glycosylated Ser
HSDGIFTDSYSRYRKQXAVKKYLAAVS

SEQ ID NO: 14 - protein, 28 aa, synthetic construct
  REGION 1..28: note = VIP glycopeptide analog
  SITE 2: note = MISC_FEATURE - D-Ser or L-Ser
  SITE 25: note = MISC_FEATURE - Glycosylated Ser
HSDAVFTDNYTRLRKQMAVKKYLNSILN

SEQ ID NO: 15 - protein, 28 aa, synthetic construct
  REGION 1..28: note = VIP glycopeptide analog
  SITE 2: note = MISC_FEATURE - D-Ser or L-Ser
  SITE 17: note = MISC_FEATURE - Nle
HSDAVFTDNYTRLRKQXAVKKYLNSILN

SEQ ID NO: 16 - protein, 28 aa, synthetic construct
  REGION 1..28: note = VIP glycopeptide analog
  SITE 2: note = MISC_FEATURE - D-Ser or L-Ser
  SITE 17: note = MISC_FEATURE - Nle
  SITE 25: note = MISC_FEATURE - Glycosylated Ser
HSDAVFTDNYTRLRKQXAVKKYLNSILN

SEQ ID NO: 17 - protein, 28 aa, synthetic construct
  REGION 1..28: note = PACAP glycopeptide analog
  SITE 28: note = MISC_FEATURE - Ser(Lactose)
HSDGIFTDSYSRYRKQLAVKKYLAAVLS

SEQ ID NO: 18 - protein, 28 aa, synthetic construct
  REGION 1..28: note = PACAP glycopeptide analog
  SITE 28: note = MISC_FEATURE - Ser(Melibiose)
HSDGIFTDSYSRYRKQLAVKKYLAAVLS

SEQ ID NO: 19 - protein, 28 aa, synthetic construct
  REGION 1..28: note = PACAP glycopeptide analog
  SITE 28: note = MISC_FEATURE - Ser(Cellobiose)
HSDGIFTDSYSRYRKQLAVKKYLAAVLS

SEQ ID NO: 20 - protein, 28 aa, synthetic construct
  REGION 1..28: note = PACAP glycopeptide analog
  SITE 2: note = MISC_FEATURE - D-Ser
  SITE 17: note = MISC_FEATURE - Norvaline
  SITE 28: note = MISC_FEATURE - L-Serine-beta-D-Glucoside
HSDGIFTDSYSRYRKQXAVKKYLAAVLS

SEQ ID NO: 21 - protein, 27 aa, synthetic construct
  REGION 1..27: note = PACAP glycopeptide analog
  SITE 2: note = MISC_FEATURE - D-Ser
  SITE 17: note = MISC_FEATURE - Norvaline
  SITE 27: note = MISC_FEATURE - L-Serine-beta-D-Glucoside
HSDGIFTDSYSRYRKQXAVKKYLAAVS

SEQ ID NO: 22 - protein, 27 aa, synthetic construct
  REGION 1..27: note = PACAP glycopeptide analog
  SITE 2: note = MISC_FEATURE - D-Ser
  SITE 17: note = MISC_FEATURE - Norvaline
  SITE 26: note = MISC_FEATURE - L-Serine-beta-D-Glucoside
HSDGIFTDSYSRYRKQXAVKKYLAASL

SEQ ID NO: 23 - protein, 28 aa, synthetic construct
  REGION 1..28: note = PACAP glycopeptide analog
  SITE 2: note = MISC_FEATURE - D-Ser
  SITE 17: note = MISC_FEATURE - Norvaline
  SITE 28: note = MISC_FEATURE - L-Serine-beta-D-Lactoside
HSDGIFTDSYSRYRKQXAVKKYLAAVLS

SEQ ID NO: 24 - protein, 27 aa, synthetic construct
  REGION 1..27: note = PACAP glycopeptide analog
  SITE 2: note = MISC_FEATURE - D-Ser
  SITE 17: note = MISC_FEATURE - Norvaline
  SITE 27: note = MISC_FEATURE - L-Serine-beta-D-Lactoside
HSDGIFTDSYSRYRKQXAVKKYLAAVS

SEQ ID NO: 25 - protein, 27 aa, synthetic construct
  REGION 1..27: note = PACAP glycopeptide analog
  SITE 2: note = MISC_FEATURE - D-Ser
  SITE 17: note = MISC_FEATURE - Norvaline
  SITE 26: note = MISC_FEATURE - L-Serine-beta-D-Lactoside
HSDGIFTDSYSRYRKQXAVKKYLAASL

SEQ ID NO: 26 - protein, 28 aa, synthetic construct
  SITE 17: note = Norleucine
HSDGIFTDSYSRYRKQXAVKKYLAAVLS

SEQ ID NO: 27 - protein, 28 aa, synthetic construct
  SITE 17: note = Norleucine
  MOD_RES 28: note = L-Serine-beta-D-Glucoside
HSDGIFTDSYSRYRKQNAVKKYLAAVLS

SEQ ID NO: 28 - protein, 28 aa, synthetic construct
  SITE 17: note = Norleucine
  MOD_RES 28: note = L-Serine-beta-D-Lactoside
HSDGIFTDSYSRYRKQXAVKKYLAAVLS

SEQ ID NO: 29 - protein, 28 aa, synthetic construct
  SITE 17: note = Norleucine
  MOD_RES 28: note = L-Serine-beta-D-Glucoside
  MOD_RES 24: note = L-Serine-beta-D-Glucoside
HSDGIFTDSYSRYRKQXAVKKYLSAVLS

SEQ ID NO: 30 - protein, 24 aa, synthetic construct
  SITE 17: note = Norleucine
HSDGIFTDSYSRYRKQXAVKKYLS

SEQ ID NO: 31 - protein, 24 aa, synthetic construct
  SITE 17: note = Norleucine
  MOD_RES 24: note = L-Serine-beta-D-Glucoside
HSDGIFTDSYSRYRKQXAVKKYLS

SEQ ID NO: 32 - protein, 24 aa, synthetic construct
  SITE 17: note = Norleucine
  MOD_RES 24: note = L-Serine-beta-D-Lactoside
HSDGIFTDSYSRYRKQXAVKKYLS

SEQ ID NO: 33 - protein, 25 aa, synthetic construct
  SITE 17: note = Norleucine
  MOD_RES 24: note = L-Serine-beta-D-Glucoside
  MOD_RES 25: note = L-Serine-beta-D-Glucoside
HSDGIFTDSYSRYRKQXAVKKYLSS

SEQ ID NO: 34 - protein, 24 aa, synthetic construct
  SITE 17: note = Norleucine
  MOD_RES 24: note = L-Serine-beta-D-Glucoside
  MOD_RES 24: note = L-Serine-beta-D-Glucoside
HSDGIFTDSYSRYRKQXAVSKYLS

SEQ ID NO: 35 - protein, 20 aa, synthetic construct
  SITE 17: note = Norleucine
HSDGIFTDSYSRYRKQXAVS

SEQ ID NO: 36 - protein, 20 aa, synthetic construct
  SITE 17: note = Norleucine
  MOD_RES 20: note = L-Serine-beta-D-Glucoside
HSDGIFTDSYSRYRKQXAVS

SEQ ID NO: 37 - protein, 20 aa, synthetic construct
  SITE 17: note = Norleucine
  MOD_RES 20: note = L-Serine-beta-D-Lactoside
HSDGIFTDSYSRYRKQXAVS

SEQ ID NO: 38 - protein, 21 aa, synthetic construct
  SITE 17: note = Norleucine
  MOD_RES 20: note = L-Serine-beta-D-Glucoside
  MOD_RES 21: note = L-Serine-beta-D-Glucoside
HSDGIFTDSYSRYRKQXAVSS

SEQ ID NO: 39 - protein, 20 aa, synthetic construct
  SITE 17: note = Norleucine
  MOD_RES 20: note = L-Serine-beta-D-Glucoside
  MOD_RES 15: note = L-Serine-beta-D-Glucoside
HSDGIFTDSYSRYRSQXAVS

SEQ ID NO: 40 - protein, 15 aa, synthetic construct
HSDGIFTDSYSRYRS

SEQ ID NO: 41 - protein, 15 aa, synthetic construct
  MOD_RES 15: note = L-Serine-beta-D-Glucoside
HSDGIFTDSYSRYRS

SEQ ID NO: 42 - protein, 15 aa, synthetic construct
  MOD_RES 15: note = L-Serine-beta-D-Lactoside
HSDGIFTDSYSRYRS

SEQ ID NO: 43 - protein, 16 aa, synthetic construct
  MOD_RES 15: note = L-Serine-beta-D-Glucoside
  MOD_RES 14: note = L-Serine-beta-D-Glucoside
HSDGIFTDSYSRYRSS

SEQ ID NO: 44 - protein, 15 aa, synthetic construct
  MOD_RES 15: note = L-Serine-beta-D-Glucoside
  MOD_RES 11: note = L-Serine-beta-D-Glucoside
HSDGIFTDSYSRYRS

SEQ ID NO: 45 - protein, 11 aa, synthetic construct
HSDGIFTDSYS

SEQ ID NO: 46 - protein, 11 aa, synthetic construct
  MOD_RES 11: note = L-Serine-beta-D-Glucoside
HSDGIFTDSYS

SEQ ID NO: 47 - protein, 11 aa, synthetic construct
  MOD_RES 11: note = L-Serine-beta-D-Lactoside
HSDGIFTDSYS

SEQ ID NO: 48 - protein, 12 aa, synthetic construct
  MOD_RES 11: note = L-Serine-beta-D-Glucoside
  MOD_RES 12: note = L-Serine-beta-D-Glucoside
HSDGIFTDSYSS

SEQ ID NO: 49 - protein, 9 aa, synthetic construct
HSDGIFTDS

SEQ ID NO: 50 - protein, 9 aa, synthetic construct
  MOD_RES 9: note = L-Serine-beta-D-Glucoside
HSDGIFTDS

SEQ ID NO: 51 - protein, 9 aa, synthetic construct
  MOD_RES 9: note = L-Serine-beta-D-Lactoside
HSDGIFTDS

SEQ ID NO: 52 - protein, 10 aa, synthetic construct
  MOD_RES 9: note = L-Serine-beta-D-Glucoside
  MOD_RES 10: note = L-Serine-beta-D-Glucoside
HSDGIFTDSS